(19) United States
(12) Patent Application Publication
(10) Pub. No.: US 2019/0029869 A1
(43) Pub. Date: Jan. 31, 2019

(54) MALE INCONTINENCE APPARATUS

(71) Applicant: Timothy Huchthausen, Hampton, CT (US)

(72) Inventor: Timothy Huchthausen, Hampton, CT (US)

(21) Appl. No.: 15/664,046

(22) Filed: Jul. 31, 2017

(51) Int. Cl. A61F 5/453; A61F 5/44

(52) U.S. Cl. CPC A61F 5/453; A61F 5/4408; A61F 5/4401

(57) ABSTRACT

Apparatus which comprises an elongated sleeve shape netting member having first and second axial extremities. The first axial extremity has an open end dimensioned and configured for engagement with the penis of a user. The second axial extremity is closed. The apparatus further includes a quantity of absorbent material disposed at the closed end thereof within the said sleeve shape member whereby when the sleeve shaped member is disposed on a penis a quantity of urine discharged from that penis will be absorbed by the absorbent material in the apparatus. The rapid and easy removal thereof without contamination of surrounding clothing and further prevention of odors caused by that presence for an extended period of time is achieved.

### TECHNICAL FIELD

The present invention is generally directed to apparatus for minimizing the consequences of male urinary incontinence. More particularly, the present invention is directed to minimizing the possible appearance and odor and their consequences.

### BACKGROUND OF THE INVENTION

Urine is a waste product made as the kidneys filter the blood. Each kidney (one kidney on each side of the abdomen) sends newly made urine to the bladder through a tube called a ureter. The bladder acts like a storage site for urine. It expands to hold the urine until a person decides to urinate. Urinary incontinence is the involuntary loss of urine.

Urinary incontinence may occur as the result of weak or damaged bladder muscles, overactive bladder muscles, certain prostate conditions, and nerve damage are just some of the possible underlying causes of urinary incontinence in men. Urinary incontinence, the loss of bladder control, is a common and often embarrassing problem. The severity ranges from occasionally leaking urine when a person coughs or sneezes to having an urge to urinate that's so sudden and strong that the person cannot get to a toilet in time.

Holding urine and maintaining continence requires normal function of the renal system as well as the nervous system. Also, a person must be able to sense, understand, and respond to the urge to urinate. The process of urination involves two phases: (1) the filling and storage phase and (2) the emptying phase. During the filling and storage phase, the bladder fills with urine from the kidneys. The bladder stretches as it fills with increasing amounts of urine. A healthy nervous system responds to the stretching of the bladder by signaling the need to urinate, while also allowing the bladder to continue to fill.

Upon urination, the muscle holding the stored urine in the bladder (the sphincter muscle) relaxes, the bladder wall muscle (the detrusor) contracts, and urine passes from the bladder to the outside of the body through another tube called the urethra. The ability to fill and store urine properly requires a functional sphincter muscle to control output of urine from the bladder and a stable detrusor muscle. To empty the bladder completely, the detrusor muscle must contract appropriately to force urine out of the bladder and the sphincter must relax to allow the urine to pass out of the body.

Urinary incontinence is defined by the International Continence Society as involuntary loss of urine that is a hygienic or social problem to the individual. Some define urinary incontinence to include any involuntary loss of urine. According to the Clinical Practice Guideline issued by the Agency for Health Care Policy and Research, there are four different types of incontinence: stress, urge, mixed, and overflow. Some doctors also include functional incontinence as a fifth potential type. The treatment of urinary incontinence varies depending on the specific cause of incontinence.

The present invention is not directed to the treatment of urinary incontinence in men. Such treatment may include behavioral treatments, like bladder training and Kegel exercises, medication, surgery, or a combination of these therapies. Research is ongoing to discover new and better treatments for urinary incontinence in men.

Various approaches have been made to minimize the consequences of such incontinence. Known apparatus does not conveniently and compactly deal with this problem. From the above, it is therefore seen that there exists a need in the art to overcome the deficiencies and limitations described herein and above.

### SUMMARY OF THE INVENTION

It is an object of the present invention to provide a very compact apparatus to facilitate carrying not only a single such apparatus in addition to multiple duplicates of such apparatus so that the user may easily carry and if necessary utilize additional duplicates.

It is another object of the present invention to provide apparatus that may be easily utilized.

A further object of the present invention is to provide an apparatus that may be easily disposed.

The objects of the present invention may be attained in apparatus which comprises an elongated sleeve shaped netting member having first and second axial extremities, said first axial extremity having an open end dimensioned and configured for engagement with the penis of a user, said second axial extremity being closed, said apparatus further including a quantity of absorbent material disposed at the closed end thereof within the said sleeve shape member whereby when the sleeve shaped member is disposed on a penis a quantity of urine discharged from that penis will be absorbed by the absorbent material in the apparatus to permit rapid and easy removal thereof without contamination of surrounding clothing and further preventing odors caused by that presence for an extended period of time.

Additional features and advantages are realized through the techniques of the present invention. Other embodiments and aspects of the invention are described in detail herein and are considered a part of the claimed invention.

The recitation herein of desirable objects which are met by various embodiments of the present invention is not meant to imply or suggest that any or all of these objects are present as essential features, either individually or collectively, in the most general embodiment of the present invention or in any of its more specific embodiments.

### BRIEF DESCRIPTION OF THE DRAWINGS

The subject matter which is regarded as the invention is particularly pointed out and distinctly claimed in the concluding portion of the specification. The invention, however, both as to organization and method of practice, together with the further objects and advantages thereof, may best be understood by reference to the following description taken in connection with the accompanying drawings in which:

FIG. 1 is a schematic view of the present invention in which the elongated sleeve shaped elastic gauze material partially rolled down in preparation for installation on a penis, and

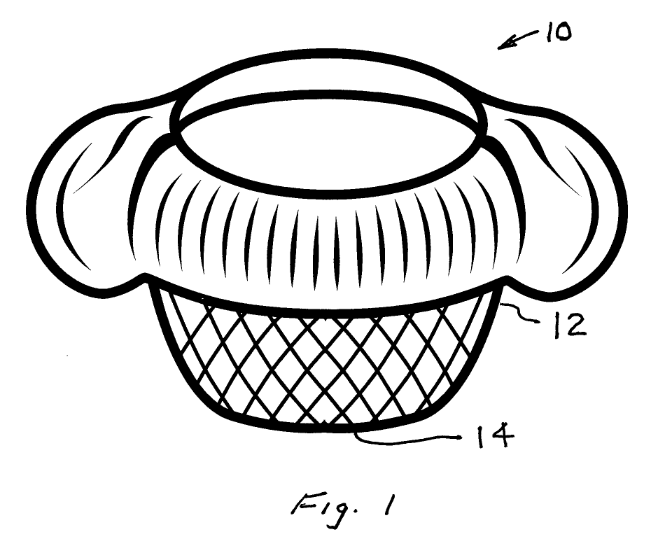

FIG. 2 is a schematic view of the same apparatus in which the elastic gauze construction has been fully unrolled and thus representative of the apparatus when installed on a penis.

### DETAILED DESCRIPTION

Apparatus of the present invention is manufactured, in part, of an elastic gauze also sometimes referred to as elastic net or tubular elastic net dressing. Examples of such products are widely available on the world wide web. Typically, elastic netting is a spandex/polyester knitted elastic net available as in a tubular form to hold dressings firmly in place and reduce dressing time. The material has a soft, open weave and does not constrict. This type of product has particular utility for use with fragile, geriatric, pediatric and at-risk skin, or those allergic to tape. The tubular form is easily cut to customize length

The natural tendency of the elastic gauze is to crumple up or reduce in size. Accordingly, the drawings included herein must be considered to be schematics in the sense that the actual apparatus will not have the same shape at all times.

The apparatus in accordance with the present invention has a shape in use that conforms to the surface on which it is disposed. More particularly the present apparatus conforms when installed to the shape of the human male penis.

While the description herein refers to the human male penis in the description and will consistently refer to that utilization, it will be understood that other embodiments of the present invention may be used for other male animals including but not limited to dogs.

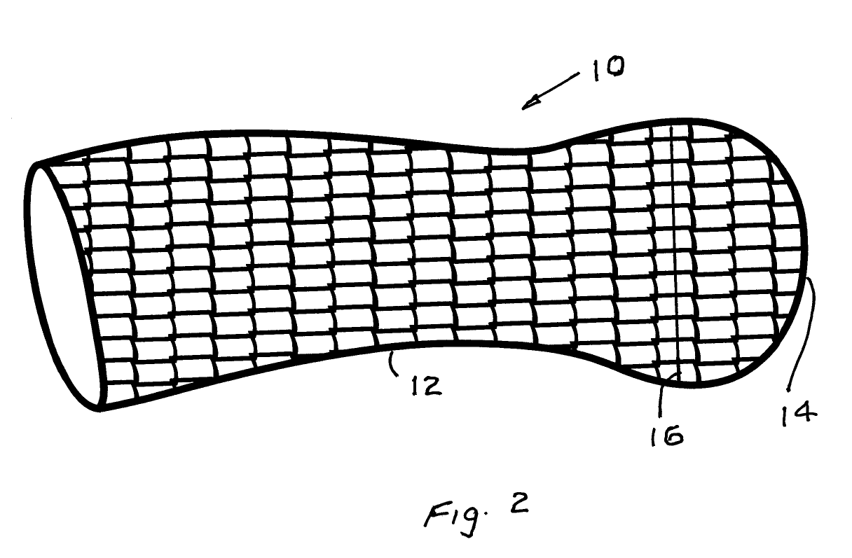

The apparatus 10 as best seen in FIG. 2 includes an elastic gauze cylindrical sleeve 12 having an elastic gauze closure at one axial extremity 14 of the generally cylindrical sleeve 12. Disposed at the axially extremity of the closed end 14 of the generally cylindrical sleeve 12 is a quantity of absorbent material 16. It will be understood that in a typical embodiment the absorbent material occupies the entire right (as viewed) axial extremity of the generally cylindrical sleeve 12. Accordingly, when the apparatus is placed on the users penis (not shown) the present apparatus 10 with the quantity of absorbent material 16 will absorb urine at the closed axial extremity of the generally cylindrical sleeve 12. Accordingly, a user after experiencing some inadvertent discharge of urine, can remove the apparatus 10 in accordance with the present invention and replace it with another identical apparatus. Because the apparatus is very compact the user can easily carry a packet containing 2. 3 or more embodiments of the apparatus 10.

All publications and patent applications mentioned in this specification are indicative of the level of skill of those skilled in the art to which this invention pertains. All publications and patent applications are herein incorporated by reference to the same extent as if each individual publication or patent application was specifically and individually indicated to be incorporated by reference.

Although the description above contains many specifics, these should not be construed as limiting the scope of the invention, but as merely providing illustrations of some of the presently preferred embodiments of this invention. Thus, the scope of this invention should be determined by the appended claims and their legal equivalents. Therefore, it will be appreciated that the scope of the present invention fully encompasses other embodiments which may become obvious to those skilled in the art, and that the scope of the present invention is accordingly to be limited by the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present invention, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. 112, sixth paragraph, unless the element is expressly recited using the phrase “means for.”

What is claimed is:

1. Apparatus which comprises an elongated sleeve shape netting member having first and second axial extremities, said first axial extremity having an open end dimensioned and configured for engagement with the penis of a user, said second axial extremity being closed, said apparatus further including a quantity of absorbent material disposed at the closed end thereof within the said sleeve shape member whereby when the sleeve-shaped member is disposed on a penis a quantity of urine discharged from that penis will be absorbed by the absorbent material in the apparatus to permit rapid and easy removal thereof without contamination of surrounding clothing and further preventing odors caused by that presence.

2. The apparatus as described in claim 1, wherein netting is a spandex/polyester knitted elastic net.
